(19) United States
(12) Patent Application Publication
(10) Pub. No.: US 2009/0111817 A1
(43) Pub. Date: Apr. 30, 2009

(54) Combinations of Eszopiclone and an Antidepressant

(75) Inventors: Judy Caron, Westwood, MA (US); Thomas Wessel, Lenox, MA (US); Karim Lalji, West Vancouver (CA); Mark A. Varney, Laguna Niguel, CA (US)

Correspondence Address: HESLIN ROTHENBERG FARLEY & MESITI P.C., 5 COLUMBIA CIRCLE, ALBANY, NY, 12203, US

(73) Assignee: SEPRACOR INC., Marlborough, MA (US)

(21) Appl. No.: 11/994,647

(22) PCT Filed: Jul. 5, 2006
(86) PCT No.: PCT/US06/26186

§ 371 Date: Jun. 6, 2008

Related U.S. Application Data
(60) Provisional application No. 60/696,976, filed on Jul. 6, 2005.

(51) Int. Cl. A61K 31/4985

(52) U.S. Cl. 514/249

(57) ABSTRACT

One aspect of the present invention relates to pharmaceutical compositions containing two or more active agents that when taken together can be used to treat, e.g., menopause, mood disorders, anxiety disorders, or cognitive disorders. The first component of the pharmaceutical composition is a sedative eszopiclone. The second component of the pharmaceutical composition is an antidepressant. The present invention also relates to a method of treating menopause, perimenopause, mood disorders, anxiety disorders, and cognitive disorders.

### FIELD OF THE INVENTION

The present invention relates to compositions and methods for the treatment of menopause and mood, anxiety, and cognitive disorders.

### BACKGROUND OF THE INVENTION

Menopause, which is caused by a lowering of the production of female sex hormones that typically occurs at around age 50, but can occur at much earlier or later ages, can generate disorders such as edema, hot flushes (or flashes), attacks of sweating, muscle and possibly joint pain, sleep disturbances, dysphoria, nervousness, mood swings, headache, palpitations (enhanced frequency of heart rate), dry mucous membranes, pain during intercourse and urinary disturbances. Hot flashes or flushing are characterized by a sudden onset of warmth in the face and neck, often progressing to the chest. Episodes generally last several minutes and are evidenced by a visible flushing of the skin. Often such episodes are accompanied by sweating, dizziness, nausea, palpitations and diaphoresis. Such symptoms can disrupt sleep and interfere with quality of life.

Although the cause of hot flashes is not completely understood, they are thought to be a disorder of thermoregulation within the hypothalamus that is a consequence of declining estrogen levels. The administration of female sex hormones, such as estrogen, is effective in palliating these symptoms, but hormone therapy is fraught with undesirable side effects. Four out of five women have disturbing menopause disorders for at least one year and 25% of women have menopause disorders for more than 5 years. Half of all women have severe disorders. Men may also have hot flashes following androgen deprivation therapy (from bilateral orchiectomy or treatment with a gonadotrophin-releasing-hormone agonist) for metastatic prostate cancer. Menopause and perimenopause may also be associated with mood disorders such as depression and anxiety.

Clinicians recognize a distinction among central nervous system illnesses, and there have been many schemes for categorizing mental disorders. The Diagnostic and Statistical Manual of Mental Disorders, Fourth Ed., Text Revision, (hereinafter, the “DSM-IV-TR™”), published by the American Psychiatric Association, and incorporated herein by reference, provides a standard diagnostic system upon which persons of skill rely. According to the framework of the DSM-IV-TR™, the CNS disorders of Axis I include: disorders diagnosed in childhood (such as, for example, attention deficit disorder or “ADD” and attention deficit/hyperactivity disorder or “ADHD”) and disorders diagnosed in adulthood. CNS disorders diagnosed in adulthood include (1) schizophrenia and psychotic disorders; (2) cognitive disorders; (3) mood disorders; (4) anxiety related disorders; (5) eating disorders; (6) substance related disorders; (7) personality disorders; and (8) “disorders not yet included” in the scheme.

Mood disorders are a group of heterogeneous, typically recurrent illnesses including unipolar (depressive) and bipolar (manic-depressive) disorders that are characterized by pervasive mood disturbances, psychomotor dysfunction, and vegetative symptoms.

In its full syndromal expression, clinical depression manifests as major depressive disorder, with episodic course and varying degrees of residual manifestations between episodes. The mood is typically depressed, irritable, and/or anxious. The patient may appear miserable, with furrowed brows, downturned corners of the mouth, slumped posture, poor eye contact, and monosyllabic (or absent) speech. The morbid mood may be accompanied by preoccupation with guilt, self-denigrating ideas, decreased ability to concentrate, indecisiveness, diminished interest in usual activities, social withdrawal, helplessness, hopelessness, and recurrent thoughts of death and suicide. Sleep disorders are common. In some, the morbid mood is so deep that tears dry up; the patient complains of an inability to experience usual emotions—including grief, joy, and pleasure—and of a feeling that the world has become colorless, lifeless, and dead.

Melancholia (formerly endogenous depression) is characterized by marked psychomotor slowing (of thinking and activity) or agitation (eg, restlessness, wringing of the hands, pressure of speech), weight loss, irrational guilt, and loss of the capacity to experience pleasure. Mood and activity vary diurnally, with a nadir in the morning. Most melancholic patients complain of difficulty falling asleep, multiple arousals, and insomnia in the middle of the night or early morning. Sexual desire is often diminished or lost. Amenorrhea can occur. Anorexia and weight loss may lead to emaciation and secondary disturbances in electrolyte balance.

In atypical depression, reverse vegetative features dominate the clinical presentation; they include anxious-phobic symptoms, evening worsening, initial insomnia, hypersomnia that often extends into the day, and hyperphagia with weight gain. Unlike patients with melancholia, those with atypical depression show mood brightening to potentially positive events but often crash into a paralyzing depression with the slightest adversity. Atypical depressive and bipolar II disorders overlap considerably.

In dysthymic disorder, depressive symptoms typically begin insidiously in childhood or adolescence and pursue an intermittent or low-grade course over many years or decades; major depressive episodes may complicate it (double depression). In pure dysthymia, depressive manifestations occur at a subthreshold level and overlap considerably with those of a depressive temperament: habitually gloomy, pessimistic, humorless, or incapable of fun; passive and lethargic; introverted; skeptical, hypercritical, or complaining; self-critical, self-reproaching, and self-derogatory; and preoccupied with inadequacy, failure, and negative events.

Thorough evaluation of many persons with depression reveals bipolar traits, and as many as one in five patients with a depressive disorder also develops frank hypomania or mania. Most switches from unipolar to bipolar disorder occur within 5 years of the onset of depressive manifestations. Predictors of a switch include early onset of depression (<25 years old), postpartum depression, frequent episodes of depression, quick brightening of mood with somatic treatments (eg, antidepressants, phototherapy, sleep deprivation, electroconvulsive therapy), and a family history of mood disorders for three consecutive generations.

Between episodes, patients with bipolar disorder exhibit depressive moodiness and sometimes high-energy activity; disruption in developmental and social functioning is more common than in unipolar disorder. In bipolar disorder, episodes are shorter (3 to 6 months), age of onset is younger, onset of episodes is more abrupt, and cycles (time from onset of one episode to that of the next) are shorter than in unipolar disorder. Cyclicity is particularly accentuated in rapid-cycling forms of bipolar disorder (usually defined as >=4 episodes/year).

In bipolar I disorder, full-fledged manic and major depressive episodes alternate. Bipolar I disorder commonly begins with depression and is characterized by at least one manic or excited period during its course. The depressive phase can be an immediate prelude or aftermath of mania, or depression and mania can be separated by months or years.

In bipolar II disorder, depressive episodes alternate with hypomanias (relatively mild, nonpsychotic periods of usually <1 week). During the hypomanic period, mood brightens, the need for sleep decreases, and psychomotor activity accelerates beyond the patient's usual level. Often, the switch is induced by circadian factors (eg, going to bed depressed and waking early in the morning in a hypomanic state). Hypersomnia and overeating are characteristic and may recur seasonally (eg, in autumn or winter); insomnia and poor appetite occur during the depressive phase. For some persons, hypomanic periods are adaptive because they are associated with high energy, confidence, and supernormal social functioning. Many patients who experience pleasant elevation of mood, usually at the end of a depression, do not report it unless specifically questioned.

Patients with major depressive episodes and a family history of bipolar disorders (unofficially called bipolar III) often exhibit subtle hypomanic tendencies; their temperament is termed hyperthymic (ie, driven, ambitious, and achievement-oriented).

In cyclothymic disorder, less severe hypomanic and mini-depressive periods follow an irregular course, with each period lasting a few days. Cyclothymic disorder is commonly a precursor of bipolar II disorder. But it can also occur as extreme moodiness without being complicated by major mood disorders. In such cases, brief cycles of retarded depression accompanied by low self-confidence and increased sleep alternate with elation or increased enthusiasm and shortened sleep. In another form, low-grade depressive features predominate; the bipolar tendency is shown primarily by how easily elation or irritability is induced by antidepressants. In chronic hypomania, a form rarely seen clinically, elated periods predominate, with habitual reduction of sleep to <6 hours. Persons with this form are constantly overcheerful, self-assured, overenergetic, full of plans, improvident, overinvolved, and meddlesome; they rush off with restless impulses and accost people.

Anxiety disorders are more common than any other class of psychiatric disorder. Panic attacks are common, affecting >⅓ of the population in a single year. Most persons recover without treatment; a few develop panic disorder. Panic disorder is uncommon, affecting <1% of the population in a 6-month period. Panic disorder usually begins in late adolescence or early adulthood and affects women two to three times more often than men. Phobic disorders involve persistent, unrealistic, yet intense anxiety that, unlike the free-floating anxiety of panic disorder, is attached to external situations or stimuli. Persons who have a phobia avoid such situations or stimuli or endure them only with great distress. However, they retain insight and recognize the excessiveness of their anxiety. In agoraphobia, anxiety about or avoidance of being trapped in situations or places with no way to escape easily if panic develops. Agoraphobia is more common than panic disorder. It affects 3.8% of women and 1.8% of men during any 6-month period. Peak age of onset is the early 20s; first appearance after age 40 is unusual. In specific phobias, clinically significant anxiety is induced by exposure to a specific situation or object, often resulting in avoidance. Specific phobias are the most common anxiety disorders but are often less troubling than other anxiety disorders. They affect 7% of women and 4.3% of men during any 6-month period.

One form of anxiety disorder is social phobia, which is a clinically significant anxiety induced by exposure to certain social or performance situations, often resulting in avoidance. Social phobias affect 1.7% of women and 1.3% of men during any 6-month period. However, more recent epidemiologic studies suggest a substantially higher lifetime prevalence of about 13%. Men are more likely than women to have the most severe form of social anxiety, avoidant personality disorder.

Yet another anxiety disorder is Obsessive-Compulsive Disorder (OCD), a disorder characterized by recurrent, unwanted, intrusive ideas, images, or impulses that seem silly, weird, nasty, or horrible (obsessions) and by urges to do something that will lessen the discomfort due to the obsessions (compulsions). Obsessive-compulsive disorder occurs about equally in men and women and affects 1.6% of the population during any 6-month period.

Posttraumatic Stress Disorder is another anxiety disorder. It is a disorder in which an overwhelming traumatic event is reexperienced, causing intense fear, helplessness, horror, and avoidance of stimuli associated with the trauma. The stressful event involves serious injury or threatened death to the person or others or actual death of others; during the event, the person experiences intense fear, helplessness, or horror. Lifetime prevalence is at least 1%, and in high-risk populations, such as combat veterans or victims of criminal violence, prevalence is reported to be between 3% and 58%.

Acute stress disorder resembles posttraumatic stress disorder in that the person has been traumatized, reexperiences the trauma, avoids stimuli that remind him of the trauma, and has increased arousal. However, by definition, acute stress disorder begins within 4 weeks of the traumatic event and lasts a minimum of 2 days but no more than 4 weeks. A person with this disorder has three or more of the following dissociative symptoms: a sense of numbing, detachment, or absence of emotional responsiveness; reduced awareness of surroundings (eg, being dazed); a feeling that things are not real; a feeling that he is not real; and amnesia for an important part of the trauma. The prevalence of acute stress disorder is unknown but is presumably proportionate to the severity of the trauma and the extent of exposure to the trauma.

Generalized Anxiety Disorder is an excessive, almost daily, anxiety and worry for ≧6 months about a number of activities or events. Generalized anxiety disorder is common, affecting 3 to 5% of the population within a 1-year period. Women are twice as likely to be affected as men. The disorder often begins in childhood or adolescence but may begin at any age.

Anxiety may be secondary to physical disorders, such as neurologic disorders (eg, brain trauma, infections, inner ear disorders), cardiovascular disorders (eg, heart failure, arrhythmias), endocrine disorders (eg, overactive adrenal or thyroid glands), and respiratory disorders (eg, asthma, chronic obstructive pulmonary disease). Anxiety may be caused by use of drugs, such as alcohol, stimulants, caffeine, cocaine, and many prescription drugs. Also, drug withdrawal is commonly associated with anxiety.

An estimated 4 to 5 million Americans (about 2% of all ages and 15% of those >age 65) have some form and degree of cognitive failure (cognitive disorder). Cognitive failure (dysfunction or loss of cognitive functions—the processes by which knowledge is acquired, retained, and used) is most commonly due to delirium (sometimes called acute confusional state) or dementia. It may also occur in association with disorders of affect, such as depression.

Delirium (Acute Confusional State) is a clinical state characterized by fluctuating disturbances in cognition, mood, attention, arousal, and self-awareness, which arises acutely, either without prior intellectual impairment or superimposed on chronic intellectual impairment. Some practitioners use the terms delirium and acute confusional state synonymously; others use delirium to refer to a subset of confused people with hyperactivity. Still others use delirium to refer to full-blown confusion and confusional state to refer to mild disorientation.

Dementia is a chronic deterioration of intellectual function and other cognitive skills severe enough to interfere with the ability to perform activities of daily living. Dementia may occur at any age and can affect young people as the result of injury or hypoxia. However, it is mostly a disease of the elderly, affecting >15% of persons >65 years old and as many as 40% of persons >80 years old. It accounts for more than half of nursing home admissions and is the condition most feared by aging adults.

Alzheimer's Disease is a progressive, inexorable loss of cognitive function associated with an excessive number of senile plaques in the cerebral cortex and subcortical gray matter, which also contains β-amyloid and neurofibrillary tangles consisting of tau protein.

Lewy body dementia may be the second most common dementia after Alzheimer's disease. Lewy bodies are hallmark lesions of degenerating neurons in Parkinson's disease and occur in dementia with or without features of Parkinson's disease. In Lewy body dementia, Lewy bodies may predominate markedly or be intermixed with classic pathologic changes of Alzheimer's disease. Symptoms, signs, and course of Lewy body dementia resemble those of Alzheimer's disease, except hallucinations (mainly visual) are more common and patients appear to have an exquisite sensitivity to antipsychotic-induced extrapyramidal adverse effects.

Cerebrovascular disease can destroy enough brain tissue to impair function. Vascular dementia, which includes impairment due to single, strategically located infarcts or to multiple small infarcts from small or medium-sized vessel disease, is more common in men and generally begins after age 70. It occurs more often in persons who have hypertension and/or diabetes mellitus or who abuse tobacco. Progressive vascular dementia can generally be slowed by controlling blood pressure, regulating blood sugar (90 to 150 mg/dL), and stopping smoking. Some degree of vascular damage is found in up to 20% of autopsies of patients with dementia.

Binswanger's dementia (subcortical arteriosclerotic encephalopathy) is uncommon and involves multiple infarcts in deep hemispheric white matter associated with severe hypertension and systemic vascular disease. Although clinically similar to vascular dementia, Binswanger's dementia may be characterized by more focal neurologic symptoms associated with acute strokes and a more rapid course of deterioration. MRI and CT show areas of leukoencephalopathy in the cerebrum semiovale adjacent to the cortex.

More than 25% of patients with Parkinson's disease have dementia; some estimates are as high as 80% (see Ch. 179). At autopsy, patients with Parkinson's disease may have some of the neuropathologic brain findings and many of the biochemical changes seen in patients with Alzheimer's disease. A less severe subcortical dementia is also associated with Parkinson's disease.

The dementia associated with progressive supranuclear palsy is commonly preceded by other neurologic symptoms, eg, multiple falls, dystonic axial rigidity, retrocollis, supranuclear ophthalmoplegia, dysphagia, and dysarthria.

Patients with Huntington's disease (chorea) may also present with symptoms of dementia, but the diagnosis is usually clarified by the family history, younger age at onset, and the disease's characteristic motor abnormalities. In case of doubt, genetic analysis can be diagnostic.

Pick's disease is a less common form of dementia, affecting the frontal and temporal regions of the cortex. Patients have prominent apathy and memory disturbances; they may show increased carelessness, poor personal hygiene, and decreased attention span. Although the clinical presentation and CT findings in Pick's disease can be quite distinctive, definitive diagnosis is possible only at autopsy. The Klüver-Bucy syndrome can occur early in the course of Pick's disease, with emotional blunting, hypersexual activity, hyperorality (bulimia and sucking and smacking of lips), and visual agnosias.

Frontal lobe dementia syndromes may result from intrinsic pathology, a primary or metastatic tumor, previous surgical manipulation, irradiation to the brain, or severe head trauma. The repeated head trauma in dementia pugilistica, which occurs in professional fighters, appears to link genetically to the 4 allele of apo E.

Normal-pressure hydrocephalus is characterized by a triad of progressive dementia, incontinence, and an unsteady, slow, and wide-based gait. Onset is usually insidious and occurs mostly in late middle or old age. The disease is more common in men and occasionally is related to prior meningitis, subarachnoid hemorrhage, head injury, or neurosurgical interventions. In most cases, evidence of precedent injury is lacking. Normal-pressure hydrocephalus may result from scarring of arachnoid villi over convexities of the brain, which results in slowed absorption of CSF (ceresbrospinal fluid), ventricular dilatation, and frontal lobe motor abnormalities. The laboratory diagnosis is based on high-normal CSF pressure (150 to 200 mm Hg) and CT evidence of ventricular dilatation and narrowed cerebral sulci at the brain's apex without widening of the subarachnoid space. The results of treatment with CSF shunting are inconsistent. The dementia is sometimes reversible; some experts recommend a therapeutic lumbar puncture to remove about 30 mL of CSF. Improvement in gait and cognition for hours or several days suggests the value of shunt placement.

Subdural hematoma can cause a change in mental status, producing coma, delirium, or a dementia syndrome. Cognitive changes may begin any time after blood begins to accumulate and can progress rapidly or slowly, according to the size and location of the hematoma. This chronic syndrome may resemble vascular dementia, with focal neurologic signs and cognitive changes. Removing the hematoma may restore function or prevent further loss of intellectual function. However, some experts believe that after hematomas have exerted pressure on the brain for a long time (perhaps a year or more), removing them does little to improve cognitive function.

The most well-known infectious cause of dementia is Creutzfeldt-Jakob disease, in which memory deficits, electroencephalographic changes, myoclonus, and sometimes ataxia are prominent. The infectious agent is a corrupted protein called a prion that can be acquired genetically, by tissue transplantation, by cannibalism, and apparently by eating products from infected cattle (with mad-cow disease). Most cases occur sporadically. It produces a characteristic spongiform encephalopathy quite different from the changes of Alzheimer's disease. The course is more rapid than that of Alzheimer's disease and usually lasts from 6 to 12 months.

Patients with Gerstmann-Sträussler-Scheinker disease, another dementia with a prion-related cause, typically present with ataxia, followed later by cognitive decline. This syndrome affects younger persons and has a longer duration than Creutzfeldt-Jakob disease.

General paresis, a form of neurosyphilis, was once a common cause of dementia in Western societies. It is still prevalent in developing countries. In addition to intellectual decline, tremors and pupillary changes can occur. The CSF is tested using the fluorescent treponemal antibody (FTA) test. A positive FTA test for syphilis establishes the diagnosis.

AIDS dementia can complicate the later stages of HIV infection. Dementia may be caused by HIV, by the JC virus that causes progressive multifocal leukoencephalopathy, or by a variety of other opportunistic infectious agents, including fungi, bacteria, viruses, or protozoa that can be identified at autopsy. Early manifestations include slowed thinking and expression, difficulty in concentration, and apathy, with preserved insight and few manifestations of depression. Motor movements are slowed; ataxia and weakness may be evident. Reflexes, including the extensor plantar responses, become abnormal. Treatment with zidovudine often induces improvement sometimes verging on the dramatic. Therefore, there exists a need to develop effective and minimally adverse therapies for the above listed disorders.

### SUMMARY OF THE INVENTION

The present invention generally relates to pharmaceutical compositions comprising eszopiclone or a pharmaceutically acceptable salt, solvate, clathrate, polymorph, or co-crystal thereof, and an antidepressant, including without limitation serotonin reuptake inhbitors, norepinephrine reuptake inhibitors, dopamine reuptake inhibitors, CRS antagonists and 5-HT_{2A} receptor modulators. The pharmaceutical compositions of the invention are useful in the treatment of menopause, perimenopause, mood disorders, anxiety disorders, and cognitive disorders.

In addition, the present invention relates to a method for augmentation of antidepressant therapy in a patient comprising administering to the patient a therapeutically effective amount of eszopiclone, or a pharmaceutically acceptable salt, solvate, clathrate, polymorph, or co-crystal thereof. The present invention also relates to a method for eliciting a dose-sparing effect in a patient undergoing treatment with an antidepressant comprising administering to the patient a therapeutically effective amount of eszopiclone, or a pharmaceutically acceptable salt, solvate, clathrate, polymorph, or co-crystal thereof.

Furthermore, the present invention relates to a method for reducing depression relapse in a patient who received antidepressant treatment comprising administering to the patient a therapeutically effective amount eszopiclone, or a pharmaceutically acceptable salt, solvate, clathrate, polymorph, or co-crystal thereof.

Co-administration of eszopiclone, a sedative agent, together with an antidepressant is beneficial in treatment of such disorders as menopause, perimenopause, mood disorders, anxiety disorders, and cognitive disorders.

### DETAILED DESCRIPTION OF THE INVENTION

The present invention relates generally to pharmaceutical compositions containing two or more active agents that when taken together have benefit in treatment of menopause, perimenopause, mood disorder, anxiety disorder, or cognitive disorder. In certain embodiments, the present invention relates to a pharmaceutical composition comprising eszopiclone and an antidepressent. In certain embodiments, the present invention relates to a pharmaceutical composition comprising eszopiclone and a serotonin reuptake inhibitor (SRI). In certain embodiments, the present invention relates to a pharmaceutical composition comprising eszopiclone and a norepinephrine reuptake inhibitor (NRI). In certain embodiments, the present invention relates to a pharmaceutical composition comprising eszopiclone and a 5-HT_{2A} modulator. In certain embodiments, the present invention relates to a pharmaceutical composition comprising eszopiclone and a dopamine reuptake inhibitor (DRI). In certain embodiments, the present invention relates to a pharmaceutical composition comprising eszopiclone and an inhibitor of reuptake of both serotonin and norepinephrine and a sedative agent. In certain embodiments, the present invention relates to a pharmaceutical composition comprising eszopiclone and an inhibitor of reuptake of three monoamines serotonin, norepinephrine and domapmine, and a sedative agent. In one embodiment, eszopiclone of the above listed embodiments is present as a pharmaceutically acceptable salt, solvate, clathrate, polymorph, or co-crystal thereof. In another embodiment, the active agent other than eszopiclone in a pharmaceutical composition of the present invention is present as a pharmaceutically acceptable salt, solvate, clathrate, polymorph, or co-crystal thereof.

Another aspect of the present invention relates to a method of treating a patient suffering from menopause, perimenopause, mood disorder, anxiety disorder, or cognitive disorder comprising the step of administering to said patient a therapeutically effective dose of a pharmaceutical composition containing two or more active agents that when taken together improve the quality of sleep or sleep disorders for said patient.

Further aspect of the present invention relates to a method of treating a patient suffering from menopause, perimenopause, mood disorder, anxiety disorder, or cognitive disorder comprising the step of administering to said patient a therapeutically effective dose of a pharmaceutical composition containing two or more active agents that when taken together improve the treatment of the patient.

In certain embodiments, said pharmaceutical composition comprises eszopiclone and a serotonin reuptake inhibitor. In certain embodiments, said pharmaceutical composition comprises eszopiclone and a norepinephrine reuptake inhibitor. In certain embodiments, said pharmaceutical composition comprises eszopiclone and a 5-HT_{2A} modulator. In certain embodiments, said pharmaceutical composition comprises eszopiclone and a dopamine reuptake inhibitor. In certain embodiments, said pharmaceutical composition comprises eszopiclone and an inhibitor of reuptake of both serotonin and norepinephrine. In certain embodiments, said pharmaceutical composition comprises eszopiclone and an inhibitor of reuptake of three monoamines serotonin, norepinephrine and domapmine, and a sedative agent. In one embodiment, the eszopiclone of the above-listed compositions is present as a pharmaceutically acceptable salt, solvate, clathrate, polymorph, or co-crystal thereof.

In another embodiment, the present invention relates to a method for augmentation of antidepressant therapy in a patient comprising administering to the patient a therapeutically effective amount of eszopiclone, or a pharmaceutically acceptable salt, solvate, clathrate, polymorph, or co-crystal thereof.

The present invention also relates to a method for eliciting a dose-sparing effect in a patient undergoing treatment with an antidepressant comprising administering to the patient a therapeutically effective amount of eszopiclone, or a pharmaceutically acceptable salt, solvate, clathrate, polymorph, or co-crystal thereof.

Furthermore, the present invention relates to a method for reducing depression relapse in a patient who received antidepressant treatment comprising administering to the patient a therapeutically effective amount of eszopiclone, or a pharmaceutically acceptable salt, solvate, clathrate, polymorph, or co-crystal thereof.

### Eszopiclone

Eszopiclone is a cyclopyrrolone that has the chemical name (+) 6-(5-chloropyrid-2-yl)-5-(4-methylpiperazin-1-yl)carbonyloxy-7-oxo-6,7-dihydro-5H-pyrrolo[3-4-b]pyrazine or (+) 6-(5-chloro-2-pyridinyl)-6,7-dihydro-7-oxo-5H-pyrrolo[3,4-b]pyrazin-5-yl 4-methylpiperazine-1-carboxylate. The chemical structure of eszopiclone is shown below:

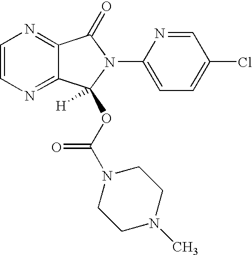

Eszopiclone is the S-(+)-optical isomer of the compound zopiclone, which is described in U.S. Pat. Nos. 6,319,926 and 6,444,673. Racemic zopiclone is described in Goa and Heel, [Drugs, 32:48-65 (1986)] and in U.S. Pat. Nos. 3,862,149 and 4,220,646. S-(+)-zopiclone, which will hereinafter be referred to by its USAN-approved generic name, eszopiclone, includes the optically pure and the substantially optically pure (e.g., 90%, 95% or 99% optical purity) S-(+)-zopiclone isomer.

Zopiclone was the first of a chemically distinct class of hypnotic and anxiolytic compounds that offers a psychotherapeutic profile of efficacy and side effects similar to the benzodiazepines. Some members of this class of compounds, the cyclopyrrolones, appear to cause less residual sedation and less slowing of reaction times than the benzodiazepines, and it offers the promise of an improved therapeutic index over benzodiazepines. Recently, the USFDA approved use of eszopiclone (LUNESTA™) for the treatment of insomnia.

Eszopiclone possesses potent activity in treating sleep disorders such as insomnia. Eszopiclone also possess potent activity in treating sleep disorders while avoiding the usual adverse effects including but not limited to drowsiness, next day effects tiredness in the morning, inability to concentrate and headache. U.S. Pat. No. 5,786,357 relates to methods of using eszopiclone also to treat convulsive disorders such as epilepsy.

The size of a prophylactic or therapeutic dose of eszopiclone in the acute or chronic management of disease will vary with the severity of the condition to be treated and the route of administration. The dose, and perhaps the dose frequency, will also vary according to the age, body weight, and response of the individual patient. In general, the total daily dose ranges, for the conditions described herein, is from about 0.25 mg to about 10 mg. Preferably, a daily dose range should be between about 0.5 mg to about 5 mg. Most preferably, a daily dose range should be between about 0.5 mg to about 3.0 mg. In one embodiment, the daily dose is 0.5 mg, 1.0 mg, 1.5 mg, 2.0 mg, 2.5 mg, or 3.0 mg. In managing the patient, the therapy may be initiated at a lower dose, perhaps about 0.5 mg to about 2 mg and increased depending-on the patient's global response. It is further recommended that children and patients over 65 years, and those with impaired renal or hepatic function, initially receive low doses, and that they be titrated based on global response and blood level. It may be necessary to use dosages outside these ranges in some cases.

In the case where an oral composition is employed, a suitable dosage range for use is from about 0.25 mg to about 10.0 mg with, in the usual case, the lower doses serving more common insomnia, and the higher doses, presented in divided dosing, reserved for control of psychiatric disorders. Preferably, a dose range of between about 0.5 mg to about 5 mg is given as a once daily administration or in divided doses if required; most preferably, a dose range of from about 0.5 mg to about 3 mg is given, either as a once daily administration or in divided doses if required. Patients may be upward titrated from below to within this dose range to a satisfactory control of symptoms as appropriate.

### Antidepressants

A considerable amount of antidepressants are known in the art. Most of the known antidepressants are inhibitors of reuptake of monoamines serotonin and/or norepinephrine. Some antidepressants are inhibitors of reuptake of dopamine. It should be noted that some antidepressants are selective inhibitors of reuptake of a specific monoamine while others produce inhibition of two or three monoamines. Therefore, a drug like clomipramine has effect on the levels of both serotonin and norepinephrine.

A large number of antidepressant agents are serotonin reuptake inhibitors (SRIs). Examples of SRI agents are: Citalopram, Duloxetine (CYMBALTA®), Escitalopram, Fluoxetine, Fluvoxamine, Milnacipran, Paroxetine, Sertraline, Clomipramine, Femoxetine, Indalpine (UPSTENE®), Alaproclate, Cericlamine, Ifoxetine, 5-HT_{2A} Modulators, MDL 100,907, SR 46349B, YM 992, Fananserin, Oxazolidine Compounds A (described in WO 98/38189), Phenylindole Compounds A (described in U.S. Pat. No. 6,486,153), Piperidinyl Compounds B (described in U.S. Pub. No. 2004/0106600), Spiroazacyclic Compounds C (described in U.S. Pub. No. 2003/0166928), and Azacyclic Compounds D (described in U.S. Pat. No. 6,756,393). Additional serotonin reuptake inhibitors contemplated for the instant invention also include buspirone, clovoxamine, cyanodothiepin, N,N-dimethyl-a-[2-(naphthalenyloxy)ethyl]benzenemethanamine (for the purposes of the present invention, this compound could be racemic, an R-enantiomer, or an S-enantiomer having a common name dapoxetine), imipramine, litoxetine, lofepramine, nefazodone, norzimeldine, trazodone, venlafaxine, viqualine, and zimeldine.

Many antidepressants are norepinephrine reuptake inhibitors (NRIs). Examples of NRI agents are: Desipramine, Maprotiline, Lofepramine, Reboxetine, Oxaprotiline, Fezolamine, Tomoxetine, and (S,S)-hydroxybupropion. The present invention also contemplates the use of norepinephrine reuptake inhibitors in general, including nortriptyline, maprotiline, protriptyline, trimipramine, venlafaxine, amitriptyline, amoxapine, doxepin, clomipramine, and lamotrigine.

Some antidepressants of the invention are dopamine reuptake inhibitirs (DRIs). Examples of DRI agents are Amineptine, Bupropion, GBR-12935, Venlafaxine (EFFEXOR®), and 2β-Propanoyl-3β-(4-tolyl)-tropane (PTT).

Antidepressants of the present invention also include atypical antidepressants. Atypical antidepressants do not possess the traditional tricyclic ring structure, but do generally share an ability to elevate synaptic monoamines, in most cases by blocking their reuptake. Bupropion, mianserin, mirtazapine, nefazadone and trazadone are examples of atypical antidepressants. Some atypical antidepressants may be classified as serotonin, norepinephrine, or dopamine reuptake inhibitors. Bupropion inhibits the dopamine and, to some extent, norepinephrine transporters. Nefazadone and trazadone display relative selectivity for the serotonin transporter. Mirtazapine appears to block α2-adrenergic autoreceptors on nerve terminals thereby increasing norepinephrine release. Mirtazepine and mianserin also block postsynaptic serotonin receptors, i.e. 5-HT_{2A}, 5-HT_{2C} and 5-HT₃ receptors.

In general, a dose of an antidepressant or a pharmaceutically acceptable salt thereof suitable for administration to a human will be in the range of 0.01 to 50 mg per kilogram body weight of the recipient per day, preferably in the range of 0.1 to 3 mg per kilogram body weight per day. Unless otherwise stated all weights of active ingredients are calculated in terms of drug per se. In certain embodiments, the desired dose is presented as two, three, four, five or more sub-doses administered at appropriate intervals throughout the day. These sub-doses may be administered in unit dosage forms, for example, containing about 5 to 50 mg.

### Combination Therapy

One aspect of the present invention relates to combination therapy. This type of therapy is advantageous because the co-administration of active ingredients achieves a therapeutic effect that is greater than the therapeutic effect achieved by administration of only a single therapeutic agent. In one embodiment, the co-administration of two or more therapeutic agents achieves a synergistic effect, i.e., a therapeutic affect that is greater than the sum of the therapeutic effects of the individual components of the combination. In another embodiment, the co-administration of two or more therapeutic agents achieves an augmentation effect.

The active ingredients that comprise a combination therapy may be administered together via a single dosage form or by separate administration of each active agent. In certain embodiments, the first and second therapeutic agents are administered in a single dosage form. The agents may be formulated into a single tablet, pill, capsule, or solution for parenteral administration and the like.

Alternatively, the first therapeutic agent and the second therapeutic agents may be administered as separate compositions, e.g., as separate tablets or solutions. The first active agent may be administered at the same time as the second active agent or the first active agent may be administered intermittently with the second active agent. The length of time between administration of the first and second therapeutic agent may be adjusted to achieve the desired therapeutic effect. In certain instances, the second therapeutic agent may be administered only a few minutes (e.g., 1, 2, 5, 10, 30, or 60 min) after administration of the first therapeutic agent. Alternatively, the second therapeutic agent may be administered several hours (e.g., 2, 4, 6, 10, 12, 24, or 36 hr) after administration of the first therapeutic agent. In certain embodiments, it may be advantageous to administer more than one dosage of the second therapeutic agent between administrations of the first therapeutic agent. For example, the second therapeutic agent may be administered at 2 hours and then again at 10 hours following administration of the first therapeutic agent. Alternatively, it may be advantageous to administer more than one dosage of the first therapeutic agent between administrations of the second therapeutic agent. Importantly, it is preferred that the therapeutic effects of each active ingredient overlap for at least a portion of the duration of each therapeutic agent so that the overall therapeutic effect of the combination therapy is attributable in part to the combined or synergistic effects of the combination therapy.

The dosage of the active agents will generally be dependent upon a number of factors including pharmacodynamic characteristics of each agent of the combination, mode and route of administration of active agent(s), the health of the patient being treated, the extent of treatment desired, the nature and kind of concurrent therapy, if any, and the frequency of treatment and the nature of the effect desired. In general, dosage ranges of the active agents often range from about 0.001 to about 250 mg/kg body weight per day. For example, for a normal adult having a body weight of about 70 kg, a dosage in the range of from about 0.1 to about 25 mg/kg body weight is typically preferred. However, some variability in this general dosage range may be required depending upon the age and weight of the subject being treated, the intended route of administration, the particular agent being administered and the like. Since two or more different active agents are being used together in a combination therapy, the potency of each agent and the interactive effects achieved using them together must be considered. Importantly, the determination of dosage ranges and optimal dosages for a particular mammal is also well within the ability of one of ordinary skill in the art having the benefit of the instant disclosure.

In certain embodiments, it may be advantageous for the pharmaceutical combination to have a relatively large amount of the first component compared to the second component. In certain instances, the ratio of the first active agent to second active agent is 30:1, 20:1, 15:1, 10:1, 9:1, 8:1, 7:1, 6:1, or 5:1. In certain embodiments, it may be preferable to have a more equal distribution of pharmaceutical agents. In certain instances, the ratio of the first active agent to the second active agent is 4:1, 3:1, 2:1, 1:1, 1:2, 1:3, or 1:4. In certain embodiments, it may be advantageous for the pharmaceutical combination to have a relatively large amount of the second component compared to the first component. In certain instances, the ratio of the second active agent to the first active agent is 30:1, 20:1, 15:1, 10:1, 9:1, 8:1, 7:1, 6:1, or 5:1. Importantly, a composition comprising any of the above-identified combinations of first therapeutic agent and second therapeutic agent may be administered in divided doses 1, 2, 3, 4, 5, 6, or more times per day or in a form that will provide a rate of release effective to attain the desired results. In a preferred embodiment, the dosage form contains both the first and second active agents. In a more preferred embodiment, the dosage form only has to be administered one time per day and the dosage form contains both the first and second active agents.

For example, a formulation intended for oral administration to humans may contain from 0.1 mg to 5 g of the first therapeutic agent and 0.1 mg to 5 g of the second therapeutic agent, both of which are compounded with an appropriate and convenient amount of carrier material varying from about 5 to about 95 percent of the total composition. Unit dosages will generally contain between from about 0.5 mg to about 1500 mg of the first therapeutic agent and 0.5 mg to about 1500 mg of the second therapeutic agent. In a preferred embodiment, the dosage comprises 0.5 mg, 1 mg, 2 mg, 3 mg, 4 mg, 5 mg, 10 mg, 25 mg, 50 mg, 100 mg, 200 mg, 300 mg, 400 mg, 500 mg, 600 mg, 800 mg, or 1000 mg, etc., up to 1500 mg of the first therapeutic agent. In a preferred embodiment, the dosage comprises 0.5 mg, 1 mg, 2 mg, 3 mg, 4 mg, 5 mg, 10 mg, 25 mg, 50 mg, 100 mg, 200 mg, 300 mg, 400 mg, 500 mg, 600 mg, 800 mg, or 1000 mg, etc., up to 1500 mg of the second therapeutic agent. The optimal ratios of the first and second therapeutic agent can be determined by standard assays known in the art.

The toxicity and therapeutic efficacy of compositions of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of RT production from infected cells compared to untreated control as determined in cell culture. Such information can be used to more accurately determine useful doses in humans. Levels in plasma may be measured, for example, by high performance liquid chromatography (HPLC).

### Synergism and Augmentation

The term “synergistic” refers to a combination which is more effective than the additive effects of any two or more single agents. A synergistic effect permits the effective treatment of a disease using lower amounts (doses) of either individual therapy. The lower doses result in lower toxicity without reduced efficacy. In addition, a synergistic effect can result in improved efficacy, e.g., improved antidepressant activity. Finally, synergy may result in an improved avoidance or reduction of disease as compared to any single therapy.

Combination therapy can allow for the use of lower doses of the first therapeutic or the second therapeutic agent (referred to as “apparent one-way synergy” herein), or lower doses of both therapeutic agents (referred to as “two-way synergy” herein) than would normally be required when either drug is used alone.

In certain embodiments, the synergism exhibited between the second therapeutic agent and the first therapeutic agent is such that the dosage of the first therapeutic agent would be sub-therapeutic if administered without the dosage of the second therapeutic agent. Alternatively, the synergism exhibited between the second therapeutic agent and the first therapeutic agent is such that the dosage of the second therapeutic agent would be sub-therapeutic if administered without the dosage of the first therapeutic agent.

The terms “augmentation” or “augment” refer to combination where one of the compounds increases or enhances therapeutic effects of another compound or compounds administered to a patient. In some instances, augmentation can result in improving the efficacy, tolerability, or safety, or any combination thereof, of a particular therapy.

In certain embodiments, the present invention relates to a pharmaceutical composition comprising a therapeutically effective dose of a first therapeutic agent together with a dose of a second therapeutic agent effective to augment the therapeutic effect of the first therapeutic agent. In other embodiments, the present invention relates to methods of augmenting the therapeutic effect in a patient of a first therapeutic agent by administering the second therapeutic agent to the patient. In other embodiments, the present invention relates to a pharmaceutical composition comprising an therapeutically effective dose of a second therapeutic agent together with a dose of a first therapeutic agent effective to augment the therapeutic effect of the second therapeutic agent. In other embodiments, the present invention relates to methods of augmenting the therapeutic effect in a patient of a second therapeutic agent by administering the first therapeutic agent to the patient.

In certain preferred embodiments, the invention is directed in part to synergistic combinations of the first therapeutic agent in an amount sufficient to render a therapeutic effect together with a second therapeutic agent. For example, in certain embodiments a therapeutic effect is attained which is at least about 2 (or at least about 4, 6, 8, or 10) times greater than that obtained with the dose of the first therapeutic agent alone. In certain embodiments, the synergistic combination provides a therapeutic effect which is up to about 20, 30 or 40 times greater than that obtained with the dose of first therapeutic agent alone. In such embodiments, the synergistic combinations display what is referred to herein as an “apparent one-way synergy”, meaning that the dose of second therapeutic agent synergistically potentiates the effect of the first therapeutic agent, but the dose of first therapeutic agent does not appear to significantly potentiate the effect of the second therapeutic agent.

In certain embodiments, the combination of active agents exhibit two-way synergism, meaning that the second therapeutic agent potentiates the effect of the first therapeutic agent, and the first therapeutic agent potentiates the effect of the second therapeutic agent. Thus, other embodiments of the invention relate to combinations of a second therapeutic agent and a first therapeutic agent where the dose of each drug is reduced due to the synergism between the drugs, and the therapeutic effect derived from the combination of drugs in reduced doses is enhanced. The two-way synergism is not always readily apparent in actual dosages due to the potency ratio of the first therapeutic agent to the second therapeutic agent. For instance, two-way synergism can be difficult to detect when one therapeutic agent displays much greater therapeutic potency relative to the other therapeutic agent.

The synergistic effects of combination therapy may be evaluated by biological activity assays. For example, the therapeutic agents are be mixed at molar ratios designed to give approximately equipotent therapeutic effects based on the EC₉₀ values. Then, three different molar ratios are used for each combination to allow for variability in the estimates of relative potency. These molar ratios are maintained throughout the dilution series. The corresponding monotherapies are also evaluated in parallel to the combination treatments using the standard primary assay format. A comparison of the therapeutic effect of the combination treatment to the therapeutic effect of the monotherapy gives a measure of the synergistic effect. Further details on the design of combination analyses can be found in B E Korba (1996) Antiviral Res. 29:49. Analysis of synergism, additivity, or antagonism can be determined by analysis of the aforementioned data using the CalcuSyn™ program (Biosoft, Inc.). This program evaluates drug interactions by use of the widely accepted method of Chou and Talalay combined with a statistically evaluation using the Monte Carlo statistical package. The data are displayed in several different formats including median-effect and dose-effects plots, isobolograms, and combination index [CI] plots with standard deviations. For the latter analysis, a CI greater than 1.0 indicates antagonism and a CI less than 1.0 indicates synergism.

Compositions of the invention present the opportunity for obtaining relief from moderate to severe cases of disease. Due to the synergistic and/or additive effects provided by the inventive combination of the first and second therapeutic agent, it may be possible to use reduced dosages of each of therapeutic agent. By using lesser amounts of other or both drugs, the side effects associated with each may be reduced in number and degree. Moreover, the inventive combination avoids side effects to which some patients are particularly sensitive.

### Formulations and Definitions

Pharmaceutical compositions of the present invention may be administered by any suitable route of administration that provides a patient with a therapeutically effective dosage of the active ingredients. Typically, the pharmaceutical compositions described herein will be formulated for oral administration or for inhalation. Suitable dosage forms include tablets, troches, cachets, caplets, capsules, including hard and soft gelatin capsules, and the like. Tablet forms, however, remain a preferred dosage form because of advantages afforded both the patient (e.g., accuracy of dosage, compactness, portability, blandness of taste and ease of administration) and to the manufacturer (e.g., simplicity and economy of preparation, stability and convenience in packaging, shipping and dispensing).

The pharmaceutical compositions may further include a “pharmaceutically acceptable inert carrier” and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques. In one embodiment, coating with hydroxypropylmethylcellulose (HPMC) is employed. “Pharmaceutically acceptable carrier” also encompasses controlled release means. Compositions of the present invention may also optionally include other therapeutic ingredients, anti-caking agents, preservatives, sweetening agents, colorants, flavors, desiccants, plasticizers, dyes, and the like. However, any such optional ingredient must be compatible with combination of active ingredients to insure the stability of the formulation.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. In one embodiment, eszopiclone is formulated as a succinate salt. In another embodiment, eszopiclone is formulated as a fumarate salt.

Eszopiclone, and many of the antidepressants, including without limitation, serotonin reuptake inhibitors, norepinephrine reuptake inhibitors, and 5-HT_{2A} modulators are chiral compounds that can exist as a racemic mixture, a non-equal mixture of enantiomers, or as a single enantiomer. Importantly, the recitation of a compound that can exist as a racemic mixture, a non-equal mixture of enantiomers, or a single enantiomer is meant to encompass all three aforementioned forms, unless stated otherwise. The term “enantiomeric excess” is well known in the art and is defined for a resolution of ab into a+b as:

$${ee}_{a} = ( \frac{conc . of a - conc . of b}{conc . of a + conc . of b} ) \times 100$$

The term “enantiomeric excess” is related to the older term “optical purity” in that both are measures of the same phenomenon. The value of e.e. will be a number from 0 to 100, zero being racemic and 100 being pure, single enantiomer. A compound which in the past might have been called 98% optically pure is now more precisely described as 96% e.e.; in other words, a 90% e.e. reflects the presence of 95% of one enantiomer and 5% of the other in the material in question. In instances when a specific enantiomer is recited (e.g., eszopiclone) for use in the compositions or methods of the present invention, this indicates that the composition contains a significantly greater proportion of the specified enantiomer in relation to the non-specified enantiomer. In a preferred embodiment, compositions comprising a specified enantiomer contain the specified enantiomer in at least 90% e.e. More preferably, such compositions comprising a specified enantiomer contain the specified enantiomer in at least 95% e.e. Even more preferably, such compositions comprising a specified enantiomer contain the specified enantiomer in at least 98% e.e. Most preferably, such compositions comprising a specified enantiomer contain the specified enantiomer in at least 99% e.e.

For example, compositions comprising eszopiclone contain the S-enantiomer of zopiclone in at least 90% e.e. More preferably, compositions comprising eszopiclone contain the S-enantiomer of zopiclone in at least 95% e.e. Even more preferably, such compositions comprising eszopiclone contain the S-enantiomer of zopiclone in at least 98% e.e. Most preferably, such compositions comprising eszopiclone contain the S-enantiomer of zopiclone in at least 99% e.e.

The term “serotonin reuptake inhibitor” refers to a compound that at least partially inhibits the reuptake of serotonin. In a preferred embodiment, the serotonin reuptake inhibitor is a selective serotonin reuptake inhibitor.

The term “selective serotonin reuptake inhibitor” refers to a compound that preferentially inhibits serotonin reuptake relative to its ability to modulate the activity of other receptors.

The term “norepinephrine reuptake inhibitor” refers to a compound that at least partially inhibits the reuptake of norepinephrine. In a preferred embodiment, the norepinephrine reuptake inhibitor is a selective norepinephrine reuptake inhibitor.

The term “selective norepinephrine reuptake inhibitor” refers to a compound that preferentially inhibits norepinephrine reuptake relative to its ability to modulate the activity of other receptors.

The term “dopamine reuptake inhibitor” refers to a compound that at least partially inhibits the reuptake of dopamine.

The term “5-HT_{2A} modulator” refers to a compound that modulates the activity of 5-HT_{2A} receptor. The term “5-HT_{2A} modulator” includes 5-HT_{2A} antagonists and 5-HT₂A inverse agonists and 5-HT_{2A} partial agonists.

The term “antagonist” refers to a compound that binds to a receptor binding site, but does not activate the receptor, a compound that binds to a receptor and blocks receptor binding site, or a compound that binds to an allosteric site on a receptor (non-competitive antagonist) resulting in prevention of activation of the receptor by its ligand. The resulting inhibition of the receptor may vary in degree and duration.

The term “patient” refers to a mammal in need of a particular treatment. In a preferred embodiment, a patient is a primate, canine, feline, or equine. In another preferred embodiment, a patient is a human.

The terms “co-administration” and “co-administering” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent at the same time.

The term “solvate” refers to a pharmaceutically acceptable form of a specified compound, with one or more solvent molecules, that retains the biological effectiveness of such compound. Examples of solvates include compounds of the invention in combination with solvents such, for example, water (to form the hydrate), isopropanol, ethanol, methanol, dimethyl sulfoxide, ethyl acetate, acetic acid, ethanolamine, or acetone. Also included are formulations of solvate mixtures such as a compound of the invention in combination with two or more solvents.

The term “antidepressant” refers to compounds used to treat depression, including without limitation: tricyclic antidepressents, such as clomipramine, amoxapine, nortriptyline, moprotilene, trimipramine, imipramine, or protriptyline; monoamine oxidase inhibitors; serotonin reuptake inhibitors, including selective serotonin reuptake inihibitors, such as citalopram, escitalopram, duloxetine, fluoxetine, sertraline, norsertraline, paroxetine, mirtrazepine, fluvoxamine, milnacipran, clominpramine, femoxetine, indapline, alaprolclate, cericlamine, or ifoxetine; norepinephrine reuptake inhibitors, including selective norepinephrine reuptake inhibitors, such as desipramine, maprotiline, lofepramine, reboxetine, oxaprotiline, fezolamine, tomoxetine, or (S,S)-hydroxybupropion; dopamine reuptake inhibitors, such as amineptine, bupropion, and venlafaxine; and atypical antidepressants, such as venlafaxine, nefazodone, or trazodone; therapeutically active isomers or metabolites of any of the foregoing; and pharmaceutically acceptable salts, solvates, clathrates, polymorphs, or co-crystals of any one of the foregoing.

The term “disorders” as used herein includes menopause, perimenopause, mood disorders, anxiety disorders, and cognitive disorders.

The term “menopause” as used herein includes various symptoms of menopause and perimenopause, such as hot flashes, awakenings due to hot flashes, nocturnal awakenings, and mood disorders associated with menopause or perimenopause, such as depression and anxiety.

The term “mood disorder” as used herein includes major depression, major depressive disorder, mild depression, severe depression without psychosis, severe depression with psychosis, melancholia (formerly endogenous depression), atypical depression, dysthymic disorder, manic depression, bipolar disorder, bipolar I disorder, bipolar II disorder, bipolar III disorder, cyclothymic disorder, and chronic hypomania.

The term “mood disorder” as used herein also includes premenstrual syndrome (PMS), premenstrual dysphoric disorder (PMDD), prenatal depression, and postpartum depression.

The term “anxiety disorder” as used herein refers to panic attacks, panic disorder, phobic disorders (such as agoraphobia, specific phobias, social phobia, avoidant personality disorder), obsessive-compulsive disorder (OCD), posttraumatic stress disorder, acute stress disorder, and generalized Anxiety Disorder.

The term “cognitive disorder” as used herein refers to delirium (acute confusional state), dementia, Alzheimer's Disease, Lewy body dementia, vascular dementia, Binswanger's dementia (subcortical arteriosclerotic encephalopathy), Parkinson's disease, progressive supranuclear palsy, Huntington's disease (chorea), Pick's disease, Klüver-Bucy syndrome, frontal lobe dementia syndromes, normal-pressure hydrocephalus, subdural hematoma, Creutzfeldt-Jakob disease, Gerstmann-Sträussler-Scheinker disease, general paresis, and AIDS dementia. The term “cognitive disorder” as used herein also includes decreased cognitive function and memory loss.

The term “treating” when used in connection with the disorders means amelioration, prevention or relief from the symptoms and/or effects associated with these disorders and includes the prophylactic administration of the compositions of the invention, or pharmaceutically acceptable salt thereof, to substantially diminish the likelihood or seriousness of the condition.

The invention now being generally described, it will be more readily understood by reference to the following examples, which are included merely for purposes of illustration of certain aspects and embodiments of the present invention, and are not intended to limit the invention.

### EXAMPLES

### Example 1

### Formulations

The following formulations are exemplary of eszopiclone and antidepressant combination tablet or capsule formulations:

TABLE 1
Eszopiclone and Fluoxetine Compositions

| | Combo Strengths | | |
|---|---|---|---|
| | (Eszopiclone/ | | |
| Ingredient | Fluoxetine, mg/unit) | | |
| (Tablet and Capsule) | 3.0/10.0 | 3.0/20.0 | 3.0/40.0 |
| Eszopiclone | 3.00 | 3.00 | 3.0 |
| Fluoxetine HCL¹ | 11.20 | 22.40 | 44.8 |
| Microcrystalline Cellulose NF (Avicel ® | 198.90 | 198.90 | 198.9 |
| PH102) | | | |
| Dibasic Calcium Phosphate Anhydrous | 90.00 | 90.00 | 90.0 |
| USP | | | |
| Croscarmellose Sodium NF | 6.00 | 6.00 | 6.0 |
| Colloidal Silicon Dioxide NF | 0.60 | 0.60 | 0.6 |
| Magnesium Stearate NF | 1.50 | 1.50 | 1.5 |
| Total tablet wt. or capsule fill wt. | 311.20 | 324.40 | 344.8 |
| Empty Size 0 hard gelatin capsule wt. | 90.00 | 90.00 | 90.00 |
| Total filled-capsule wt. | 401.20 | 414.40 | 434.80 |

¹Fluoxetine HCL potency is expressed in terms of Fluoxetine free base.
1.12 mg Fluoxetine HCL is equivalent to 1.00 mg Fluoxetine of free base.

TABLE 2
Eszopiclone and Sertraline Compositions

| | Combo Strengths | | |
|---|---|---|---|
| | (Eszopiclone/ | | |
| Ingredient | Sertraline, mg/unit) | | |
| (Tablet and Capsule) | 3.0/25.0 | 3.0/50.0 | 3.0/100.0 |
| Eszopiclone | 3.00 | 3.00 | 3.00 |
| Sertraline HCL¹ | 28.00 | 56.00 | 112.00 |
| Microcrystalline Cellulose, NF (Avicel ® | 198.90 | 198.90 | 198.90 |
| PH102) | | | |
| Dibasic Calcium Phosphate Anhydrous | 90.00 | 90.00 | 90.00 |
| USP | | | |
| Croscarmellose Sodium, NF | 6.00 | 6.00 | 6.00 |
| Colloidal Silicon Dioxide, NF | 0.60 | 0.60 | 0.60 |
| Magnesium Stearate, NF | 1.50 | 1.50 | 1.50 |
| Total tablet wt. or capsule fill wt. | 328.00 | 356.00 | 412.00 |
| Empty Size 0 hard gelatin capsule wt. | 90.00 | 90.00 | 90.00 |
| Total weight of filled capsule | 418.00 | 446.00 | 502.00 |

¹Sertraline HCL the potency is expressed in terms of Sertraline free base.
1.12 mg Sertraline HCL is equivalent to 1.00 mg Sertraline of free base.

TABLE 3
Eszopiclone and Escitalopram Compositions

| | Combo Strengths | | |
|---|---|---|---|
| | (Eszopiclone/ | | |
| Ingredient | Escitalopram, mg/unit) | | |
| (Tablet and Capsule) | 3.0/5.0 | 3.0/10.0 | 3.0/20.0 |
| Eszopiclone | 3.00 | 3.00 | 3.00 |
| Escitalopram Oxalate¹ | 6.40 | 12.80 | 25.60 |
| Microcrystalline Cellulose, NF (Avicel ® | 198.90 | 198.90 | 198.90 |
| PH102) | | | |
| Dibasic Calcium Phosphate Anhydrous | 90.00 | 90.00 | 90.00 |
| USP | | | |
| Croscarmellose Sodium, NF | 6.00 | 6.00 | 6.00 |
| Colloidal Silicon Dioxide, NF | 0.60 | 0.60 | 0.60 |
| Magnesium Stearate, NF | 1.50 | 1.50 | 1.50 |
| Total tablet wt. or capsule fill wt. | 306.40 | 312.80 | 325.60 |
| Empty Size 0 hard gelatin capsule wt. | 90.00 | 90.00 | 90.00 |
| Total weight of filled-capsule | 396.40 | 402.80 | 415.60 |

¹Escitalopram Oxalate the potency is expressed in terms of Escitalopram free base.
1.28 mg Escitalopram Oxalate is equivalent to 1.00 mg Escitalopram free base.

TABLE 4
Eszopiclone and Duloxetine Compositions

| | Combo Strengths | | |
|---|---|---|---|
| | Eszopiclone/ | | |
| | Duloxetine, mg/unit | | |
| Ingredient | 3.0 mg/ | | |
| (Tablet and Capsule) | 20.0 | 3.0/30.0 | 3.0/60.0 |
| Eszopiclone | 3.00 | 3.00 | 3.00 |
| Duloxetine HCL¹ | 22.40 | 33.60 | 67.20 |
| Microcrystalline Cellulose NF (Avicel ® | 198.90 | 198.90 | 198.90 |
| PH102) | | | |
| Dibasic Calcium Phosphate Anhydrous | 90.00 | 90.00 | 90.00 |
| USP | | | |
| Croscarmellose Sodium NF | 6.00 | 6.00 | 6.00 |
| Colloidal Silicon Dioxide NF | 0.60 | 0.60 | 0.60 |
| Magnesium Stearate NF | 1.50 | 1.50 | 1.50 |
| Total tablet wt. or capsule fill wt. | 322.40 | 333.60 | 367.20 |
| Empty Size 0 hard gelatin capsule wt. | 90.00 | 90.00 | 90.00 |
| Total filled-capsule wt.) | 412.40 | 423.60 | 457.20 |

¹Duloxetine HCL potency is expressed in terms of Duloxetine free base.
1.12 mg Duloxetine HCL is equivalent to 1.00 mg Duloxetine free base.

TABLE 5
Eszopiclone and Paroxetine Compositions

| | Combo Strengths | |
|---|---|---|
| | (Eszopiclone/Paroxetine, | |
| Ingredient | mg/unit) | |
| (Tablet and Capsule) | 3.0/10.0 | 3.0/40.0 |
| Eszopiclone | 3.00 | 3.00 |
| Paroxetine HCL hemi hydrate¹ | 11.40 | 45.60 |
| Microcrystalline Cellulose NF (Avicel ® | 198.90 | 198.90 |
| PH102) | | |
| Dibasic Calcium Phosphate Anhydrous | 90.00 | 90.00 |
| USP | | |
| Croscarmellose Sodium NF | 6.00 | 6.00 |
| Colloidal Silicon Dioxide NF | 0.60 | 0.60 |
| Magnesium Stearate NF | 1.50 | 1.50 |
| Total tablet wt. or capsule fill wt. | 311.40 | 345.60 |
| Empty Size 0 hard gelatin capsule wt. | 90.00 | 90.00 |
| Total filled-capsule wt. | 401.40 | 435.60 |

¹Paroxetine HCL potency is expressed in terms of Paroxetine free base.
1.14 mg Paroxetine HCL hemi hydrate is equivalent to 1.00 mg Paroxetine free base.

TABLE 6
Eszopiclone and S-Fluoxetine Compositions

| | Combo Strengths | | |
|---|---|---|---|
| | (Eszopiclone/ | | |
| Ingredient | S-Fluoxetine, mg/unit) | | |
| (Tablet and Capsule) | 3.0/10.0 | 3.0/20.0 | 3.0/40.0 |
| Eszopiclone | 3.0 | 3.0 | 3.0 |
| S-Fluoxetine HCL¹ | 11.2 | 22.4 | 44.8 |
| Macrocrystalline Cellulose NF (Avicel ® | 198.9 | 198.9 | 198.9 |
| PH102) | | | |
| Dibasic Calcium Phosphate Anhydrous | 90.0 | 90.0 | 90.0 |
| USP | | | |
| Croscarmellose Sodium NF | 6.0 | 6.0 | 6.0 |
| Colloidal Silicon Dioxide NF | 0.6 | 0.6 | 0.6 |
| Magnesium Stearate NF | 1.5 | 1.5 | 1.5 |
| Total tablet wt. or capsule fill wt. | 311.2 | 324.4 | 344.80 |
| Empty Size 0 hard gelatin capsule wt. | 90.0 | 90.0 | 90.0 |
| Total filled-capsule wt. | 401.2 | 411.4 | 434.80 |

²S-Fluoxetine HCL potency is expressed in terms of Fluoxetine free base. 1.12 mg S-Fluoxetine HCL is equivalent to 1.00 mg Fluoxetine free base.

The above-presented formulations may be prepared by performing the following steps:

1. Screen eszopiclone API (active pharmaceutical ingredient) through 80 mesh.

2. Screen antidepressant API (active pharmaceutical ingredient) through 40 mesh.

3. Screen remaining ingredients through # 20 or # 30 mesh screen.

4. Blend eszopiclone API with a portion of MCC (microcrystalline cellulose).

5. Blend antidepressant API with the blend from Step 4.

6. Blend the mixture from Step 5 with remaining MCC in three steps.

7. Blend API:MCC mixture from Step 6 with dicalcium phosphate.

8. Mix croscarmellose with silicon dioxide, then blend with the mixture from Step 7.

9. Blend mixture from Step 8 with magnesium stearate.

10. For tablets, compress on a suitable tablet press machine.

11. For capsules, fill into Size 0 hard gelatin capsules on a suitable capsule filling machine.

12. For tablets, coat the tablet cores from Step 10 with Opadry II in a suitable conventional tablet coating machine.

### Example 2

### Clinical Study on Treatment of Menopause or Perimenopause with Eszopiclone

The study was aimed at observing efficacy of eszopiclone 3 mg compared to placebo in the treatment of insomnia secondary to perimenopause or menopause. The study was a multicenter, randomized, double-blind, placebo-controlled, parallel-group study. The study had a one-week single-blind placebo run-in period, followed by four weeks of double blind treatment, and one week of single blind placebo wash-out. The primary method of analysis compared the post-randomization results between the two treatment groups.

Subjects were women with insomnia secondary to perimenopause or menopause. Subjects were perimenopausal or menopausal and had insomnia symptoms including ≧45 minutes sleep latency (SL) and total sleep time (TST)≦6 hours. Perimenopausal/menopausal symptoms predated the onset of sleep disturbance symptoms. The patient population was predominately Caucasian (77.2%). The mean age was 49, with a range of 40-60.

A total of 410 subjects were randomized. Among them, 201 received 3 mg of eszopiclone (ESZ) nightly (at bedtime) for four weeks and 209 received matching placebo (PBO). The discontinuation rates were moderate, 11.9% in the ESZ group and 12.9% in the PBO group.

The ESZ group had significantly fewer nocturnal awakenings due to hot flashes during Week 1 compared with PBO (LS means of 0.3 and 0.5 per night for ESZ and PBO, respectively; p=0.0016). This effect was not significant for the other weeks, but was marginally significant for the DB average (p=0.059). When change from baseline was analyzed, ESZ significantly reduced the number of nocturnal awakenings due to hot flashes in Week 1 compared with PBO (p<0.0001). The difference was not significant for Week 2, but was marginally significant for Weeks 3 and 4 (p=0.094 and 0.055, respectively) and was significant for the DB average (p=0.0045). See Table 7.

TABLE 7
Number of Nocturnal Awakenings due to Hot Flashes (Intent-to-Treat Population)

| | | Placebo | | Eszopiclone 3 mg | |
|---|---|---|---|---|---|
| Time | | Observed | Change from | Observed | Change from |
| Point | Statistic | Value | Baseline [1] | Value | Baseline [1] |
| Baseline | N | 171 | | 150 | |
| | Mean (SD) | 1.1 (1.2) | | 1.3 (1.2) | |
| | 25th Percentile | 0.0 | | 0.3 | |
| | Median | 1.0 | | 1.0 | |
| | 75th Percentile | 1.5 | | 2.0 | |
| | Minimum, Maximum | 0.0, 10.0 | | 0.0, 6.0 | |
| Week 1 | N | 179 | 157 | 175 | 140 |
| | Mean (SD) | 0.8 (1.0) | −0.2 (0.9) | 0.5 (0.7) | −0.7 (1.0) |
| | 25th Percentile | 0.0 | −0.7 | 0.0 | −1.2 |
| | Median | 0.5 | 0.0 | 0.2 | −0.5 |
| | 75th Percentile | 1.3 | 0.2 | 1.0 | 0.0 |
| | Minimum, Maximum | 0.0, 5.0 | −5.0, 2.3 | 0.0, 3.0 | −6.0, 0.8 |
| | Least Squares Means (SE) | 0.8 (0.1) | | 0.5 (0.1) | |
| | [2] | | | | |
| | p-value vs. placebo [2] | | | <.0001 | |
| | Least Squares Means (SE) | | −0.3 (0.1) | | −0.7 (0.1) |
| | [3] | | | | |
| | p-value vs. placebo [3] | | | | <.0001 |
| Week 2 | N | 174 | 153 | 170 | 139 |
| | Mean (SD) | 0.6 (0.8) | −0.5 (1.0) | 0.5 (0.7) | −0.7 (1.0) |
| | 25th Percentile | 0.0 | −1.0 | 0.0 | −1.0 |
| | Median | 0.3 | −0.4 | 0.0 | −0.6 |
| | 75th Percentile | 1.0 | 0.0 | 1.0 | 0.0 |
| | Minimum, Maximum | 0.0, 4.3 | −6.8, 1.3 | 0.0, 3.0 | −6.0, 2.0 |
| | Least Squares Means (SE) | 0.6 (0.1) | | 0.5 (0.1) | |
| | [2] | | | | |
| | p-value vs. placebo [2] | | | 0.2137 | |
| | Least Squares Means (SE) | | −0.5 (0.1) | | −0.6 (0.1) |
| | [3] | | | | |
| | p-value vs. placebo [3] | | | | 0.1963 |
| Week 3 | N | 162 | 147 | 164 | 129 |
| | Mean (SD) | 0.6 (0.8) | −0.5 (1.0) | 0.5 (0.7) | −0.7 (1.1) |
| | 25th Percentile | 0.0 | −0.8 | 0.0 | −1.0 |
| | Median | 0.3 | −0.3 | 0.0 | −0.4 |
| | 75th Percentile | 1.0 | 0.0 | 1.0 | 0.0 |
| | Minimum, Maximum | 0.0, 4.6 | −6.2, 2.7 | 0.0, 3.0 | −6.0, 1.5 |
| | Least Squares Means (SE) | 0.6 (0.1) | | 0.5 (0.1) | |
| | [2] | | | | |
| | p-value vs. placebo [2] | | | 0.1583 | |
| | Least Squares Means (SE) | | −0.5 (0.1) | | −0.6 (0.1) |
| | [3] | | | | |
| | p-value vs. placebo [3] | | | | 0.2408 |
| Week 4 | N | 151 | 135 | 154 | 121 |
| | Mean (SD) | 0.6 (0.9) | −0.5 (1.0) | 0.4 (0.7) | −0.8 (1.2) |
| | 25th Percentile | 0.0 | −1.0 | 0.0 | −1.3 |
| | Median | 0.0 | −0.3 | 0.0 | −0.7 |
| | 75th Percentile | 1.0 | 0.0 | 1.0 | 0.0 |
| | Minimum, Maximum | 0.0, 5.3 | −4.8, 4.0 | 0.0, 3.6 | −6.0, 2.1 |
| | Least Squares Means (SE) | 0.6 (0.1) | | 0.4 (0.1) | |
| | [2] | | | | |
| | p-value vs. placebo [2] | | | 0.0786 | |
| | Least Squares Means (SE) | | −0.5 (0.1) | | −0.7 (0.1) |
| | [3] | | | | |
| | p-value vs. placebo [3] | | | | 0.0683 |
| DB Avg. | N | 192 | 165 | 188 | 146 |
| | Mean (SD) | 0.7 (0.8) | −0.4 (0.9) | 0.5 (0.6) | −0.7 (1.0) |
| | 25th Percentile | 0.0 | −0.8 | 0.0 | −1.1 |
| | Median | 0.4 | −0.2 | 0.2 | −0.5 |
| | 75th Percentile | 1.0 | 0.0 | 0.9 | 0.0 |
| | Minimum, Maximum | 0.0, 4.6 | −6.0, 1.5 | 0.0, 2.7 | −6.0, 1.5 |
| | Least Squares Means (SE) | 0.7 (0.1) | | 0.5 (0.1) | |
| | [2] | | | | |
| | p-value vs. placebo [2] | | | 0.0057 | |
| | Least Squares Means (SE) | | −0.4 (0.0) | | −0.7 (0.1) |
| | [3] | | | | |
| | p-value vs. placebo [3] | | | | 0.0016 |

[1] Week 1 = First week of double-blind treatment, Week 2 = Second week of double-blind treatment, etc. DB Average includes all scheduled assessments obtained after Visit 3 up to and including Visit 5. Baseline is the average of all pre-DB observations.
[2] The pairwise comparison is a two-sided test performed using an ANOVA model, using the MIXED procedure with treatment and site as fixed effects.
[3] The pairwise comparison is a two-sided test performed using an ANCOVA model, using the MIXED procedure with treatment and site as fixed effects and baseline as the covariate.

A Physician Global Assessment was administered at Week 4, the end of the double-blind treatment period. ESZ patients had significantly better scores at this time compared with PBO (LS means of 2.7 and 3.3 for ESZ and PBO, respectively; p<0.0001). See Table 8.

TABLE 8
Menopause and Perimenopause Study, Physician Global
Assessment (Intent-to-Treat Population)

| | | Placebo | | Eszopiclone 3 mg | |
|---|---|---|---|---|---|
| | | Observed | Change from | Observed | Change from |
| Visit (Week) | Statistic | Value | Baseline | Value | Baseline |
| 3 (Baseline) | N | 202 | | 195 | |
| | Mean (SD) | 3.6 (1.0) | | 3.7 (1.0) | |
| | 25th Percentile | 3.0 | | 4.0 | |
| | Median | 4.0 | | 4.0 | |
| | 75th Percentile | 4.0 | | 4.0 | |
| | Minimum, Maximum | 0.0, 6.0 | | 0.0, 7.0 | |
| 5 (Week 4) | N | 191 | 188 | 189 | 185 |
| | Mean (SD) | 3.3 (1.1) | −0.3 (1.4) | 2.6 (1.2) | −1.0 (1.4) |
| | 25th Percentile | 2.0 | −1.0 | 2.0 | −2.0 |
| | Median | 4.0 | 0.0 | 2.0 | −1.0 |
| | 75th Percentile | 4.0 | 0.0 | 4.0 | 0.0 |
| | Minimum, Maximum | 1.0, 6.0 | −4.0, 5.0 | 1.0, 6.0 | −4.0, 6.0 |
| | Least Squares Means | 3.3 (0.1) | | 2.7 (0.1) | |
| | (SE) [1] | | | | |
| | p-value vs. placebo [1] | | | <.0001 | |
| | Least Squares Means | | −0.3 (0.1) | | −0.9 (0.1) |
| | (SE) [2] | | | | |
| | p-value vs. placebo [2] | | | | <.0001 |

[1] The pairwise comparison is a two-sided test performed using an ANOVA model, using the MIXED procedure with treatment and site as fixed effects.
[2] The pairwise comparison is a two-sided test performed using an ANCOVA model, using the MIXED procedure with treatment and site as fixed effects and baseline as the covariate.
Note(s):
The responses to the assessment question: Overall the subject's perimenopausal or menopausal symptoms since the last assessment are: 0 = Not assessed, 1 = Very much improved, 2 = Much improved, 3 = Minimally improved, 4 = No change, 5 = Minimally worse, 6 = Much worse, 7 = Very much worse.

The results of the study will change slightly because data from one site, consisting of 11 of the 410 subjects analyzed above will be excluded due to negative findings during a site audit. It is expected that the conclusions of the study will not change after exclusion of these 11 subjects.

The contents of each of the references cited herein, including the contents of the references cited within the primary references, are herein incorporated by reference in their entirety.

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

1. A pharmaceutical composition comprising eszopiclone, or a pharmaceutically acceptable salt, clathrate, polymorph, or co-crystal thereof, and an antidepressant.

2. The pharmaceutical composition of claim 1 wherein the antidepressant is a serotonin reuptake inhibitor.

3. The pharmaceutical composition of claim 2 wherein the serotonin reuptake inhibitor is citalopram, duloxetine, escitalopram, fluoxetine, fluvoxamine, milnacipran, paroxetine, sertraline, clominpramine, femoxetine, indapline, alaprolclate, cericlamine, or ifoxetine, or a pharmaceutically acceptable salt, clathrate, polymorph, or co-crystal of any one of them.

4. The pharmaceutical composition of claim 1 wherein the antidepressant is a norepinephrine reuptake inhibitor.

5. The pharmaceutical composition of claim 4 wherein said norepinephrine reuptake inhibitor is desipramine, maprotiline, lofepramine, reboxetine, oxaprotiline, fezolamine, tomoxetine, or (S,S)-hydroxybupropion, or a pharmaceutically acceptable salt, clathrate, polymorph, or co-crystal of any one of them.

6. The pharmaceutical composition of claim 1 wherein the antidepressant is a dopamine reuptake inhibitor.

7. The pharmaceutical composition of claim 1 wherein the antidepressant is a 5-HT_{2A} modulator.

8. The pharmaceutical composition of claim 7, wherein the 5-HT_{2A} modulator is a 5-HT_{2A} antagonist.

9. The pharmaceutical composition of claim 7, wherein the 5-HT_{2A} modulator is a 5-HT_{2A} inverse agonist.

10. The pharmaceutical composition of claim 7 wherein said 5 HT₂A modulator is MDL 100907, SR 46349B, YM 992, fananserin, oxazolidine compounds A, phenylindole compounds A, piperidinyl compounds B, spiroazacyclic compounds C, or azacyclic compounds D, or a pharmaceutically acceptable salt, clathrate, polymorph, or co-crystal of any one of them.

11. A method of treating a patient suffering from a menopause or perimenopause, comprising the step of co-administering to a patient in need thereof a therapeutically effective amount of eszopiclone, or a pharmaceutically acceptable salt, clathrate, polymorph, or co-crystal thereof, and a therapeutically effective amount of an antidepressant.

12. A method of treating a patient suffering from a mood disorder, comprising the step of co-administering to a patient in need thereof a therapeutically effective amount of eszopiclone, or a pharmaceutically acceptable salt, clathrate, polymorph, or co-crystal thereof, and a therapeutically effective amount of an antidepressant.

13. A method of treating a patient suffering from an anxiety disorder, comprising the step of co-administering to a patient in need thereof a therapeutically effective amount of eszopiclone, or a pharmaceutically acceptable salt, clathrate, polymorph, or co-crystal thereof, and a therapeutically effective amount of an antidepressant.

14. A method of treating a patient suffering from a cognitive disorder, comprising the step of co-administering to a patient in need thereof a therapeutically effective amount of eszopiclone, or a pharmaceutically acceptable salt, clathrate, polymorph, or co-crystal thereof, and a therapeutically effective amount of an antidepressant.

15. The method of any of claims 11 through 14, wherein the antidepressant is a serotonin reuptake inhibitor.

16. The method of any of claims 11 through 14, the antidepressant is a serotonin reuptake inhibitor is selected from citalopram, duloxetine, escitalopram, fluoxetine, fluvoxamine, milnacipran, paroxetine, sertraline, clominpramine, femoxetine, indapline, alaprolclate, cericlamine, or ifoxetine, or a pharmaceutically acceptable salt, clathrate, polymorph, or co-crystal of any one of them.

17. The method of any of claims 11 through 14, wherein the antidepressant is a norepinephrine reuptake inhibitor.

18. The method of any of claims 11 through 14, wherein the antidepressant is a norepinephrine reuptake inhibitor selected from desipramine, maprotiline, lofepramine, reboxetine, oxaprotiline, fezolamine, tomoxetine, or (S,S)-hydroxybupropion, or a pharmaceutically acceptable salt, clathrate, polymorph, or co-crystal of any one of them.

19. The method of any of claims 11 through 14, wherein the antidepressant is a dopamine reuptake inhibitor.

20. The method of any of claims 11 through 14, wherein the antidepressant is a dopamine reuptake inhibitor selected from amineptine, bupropion, GBR-12935, venlafaxine, or 2β-propanoyl-3β-(4-tolyl)-tropane, or a pharmaceutically acceptable salt, clathrate, polymorph, or co-crystal of any one of them.

21. The method of any of claims 11 through 14, wherein the antidepressant is a 5-HT_{2A} modulator.

22. The method of any of claims 11 through 14, wherein the antidepressant is a 5-HT_{2A} modulator selected from MDL 100907, SR 46349B, YM 992, fananserin, oxazolidine compounds A, phenylindole compounds A, piperidinyl compounds B, spiroazacyclic compounds C, or azacyclic compounds D, or a pharmaceutically acceptable salt, clathrate, polymorph, or co-crystal of any one of them.

23. The method claim 12, wherein the mood disorder is selected from major depression, major depressive disorder, mild depression, severe depression without psychosis, severe depression with psychosis, melancholia, atypical depression, dysthymic disorder, manic depression, bipolar disorder, bipolar I disorder, bipolar II disorder, bipolar III disorder, cyclothymic disorder, chronic hypomania, premenstrual syndrome, premenstrual dysphoric disorder, prenatal depression, and postpartum depression.

24. The method claim 13, wherein the anxiety disorder is selected from panic attacks, panic disorder, phobic disorders, obsessive-compulsive disorder, posttraumatic stress disorder, acute stress disorder, and generalized Anxiety Disorder.

25. The method claim 14, wherein the cognitive disorder is selected from delirium, dementia, Alzheimer's Disease, Lewy body dementia, vascular dementia, Binswanger's dementia, Parkinson's disease, progressive supranuclear palsy, Huntington's disease, Pick's disease, Klüver-Bucy syndrome, frontal lobe dementia syndromes, normal-pressure hydrocephalus, subdural hematoma, Creutzfeldt-Jakob disease, Gerstmann-Sträussler-Scheinker disease, general paresis, AIDS dementia, decreased cognitive function and memory loss.

26. The pharmaceutical composition of claim 6 wherein said dopamine reuptake inhibitor is amineptine, bupropion, GBR-12935, venlafaxine, or 2β-propanoyl-3β-(4-tolyl)-tropane, or a pharmaceutically acceptable salt, clathrate, polymorph, or co-crystal of any one of them.
